(19) United States
(12) Patent Application Publication
(10) Pub. No.: US 2003/0125264 A1
(43) Pub. Date: Jul. 3, 2003

(54) Methods For Treating Wounds

(75) Inventor: Sohail Malik, Roswell, GA (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/034,150

(22) Filed: Dec. 29, 2001

(51) Int. Cl. A61K 31/7048; A61K 31/353

(52) U.S. Cl. 514/27; 514/456

(57) ABSTRACT

Pharmaceutical compositions, and methods of using the same, are provided utilizing effective amounts of one or more flavones, flavonols, flavanones, isoflavanones and isoflavones to increase cell proliferation in various tissues and cell lines. As examples, the composition and methods of the present invention can be used to increase proliferation of fibroblast cells and, more particularly, in the treatment of wounds as well as strengthening of the skin.

### FIELD OF THE INVENTION

The present invention relates to methods and compositions for increasing mammalian cell proliferation.

### BACKGROUND OF THE INVENTION

Damage or injury to tissues and/or organs is a common occurrence. The body is most often able to isolate the damaged area and then repair itself by removing and replacing damaged tissue. Injury to tissues and organs can originate from a great variety of sources such as, for example, trauma, UV degradation, toxic and/or pathogenic degradation, thermal degradation (e.g. excessive heat or cold) and so forth. While the body has an impressive array of response mechanisms that limit tissue damage and promote repair, methods of increasing the speed and degree of repair are continually being sought out. In this regard, increasing the speed and/or degree that injuries are healed is beneficial in that it (i) decreases the pain and discomfort commonly associated with the wound and wound healing process; (ii) decreases the chances of developing an infection or other ailment during a period when the tissue or organ has a reduced capacity to ward of illnesses; and (iii) reduce health costs associated with treating such conditions.

In this regard, and by way of example, the skin is the largest organ in the body and, not surprisingly, wounds and injuries to the skin are a common occurrence. Healing of wounds in the skin has three general phases including (1) inflammation, migration and proliferation; (2) repair which includes the formation of collagen and other compounds; (3) wound closure. Initially, inflammatory cells and other cells migrate into and fill the damaged area. Then, in the repair phase, new connective tissues are formed from fibronectin, which in turn results in the production of collagen fibrils and eventually larger collagen fibers. The wound is thereafter closed by wound contraction which results, in part, by the modified fibroblasts present in and around the wound.

Fibroblasts, endothelial cells and keratinocytes are indispensable in cutaneous wound repair. All three cell types play vital roles in the initial phase of wound healing. Fibroblasts migrate into the wound site within about 24 hours after injury. During a later phase of healing (typically about 4-21 days), fibroblasts are activated and undergo a burst of proliferative and synthetic activity. They produce high amount of fibronectin and synthesize other proteinaceous components of extracellular matrix, including collagen, elastin and glycosaminglycans. Fibroblasts are also known to contribute in contraction of the wound. Accordingly, fibroblast proliferating agents have therefore been shown to increase the wound healing process. See, for example, S. Casadio et al., On the Healing Properties of Esters of D-panthenol with Terpene Acids, with Particular Reference to D-pantothenyl Trifarnesylacetate, Arzneimittelforschung., 17, 1122-1125, (1967); M. Aprahamian et al., Effects of Supplemental Pantothenic Acid on Wound Healing: Experimental Study in Rabbit, American Journal of Clinical Nutrition 41, 578-589 (1985); B. J. Weimann and et al., Studies on Wound Healing: Effects of Calcium D-Pantothenate on the Migration, Proliferation and Protein Synthesis of Human Dermal Fibroblasts in Culture, International Journal of Vitamin Nutrition Res., 69, 113-119, (1999).

The body produces many substances generally known as growth factors such as, for example, platelet-derived growth factor (PDGF), platelet-derived angiogenesis factor (PDAF), vascular endothelial growth factor (VEGF), platelet-derived epidermal growth factor (PDEGF), platelet factor 4 (PF4), transforming growth factor .beta. (TGF-B), transforming growth factor a (TGF-A), insulin-like growth factors 1 and 2 (IGF-1 and IGF-2), .beta. thromboglobulin-related proteins (BTG), thrombospondin (TSP), fibronectin, von Wallinbrand's factor (vWF), angiogenin, keratinocyte growth factor (KGF), epidermal growth factor (EGF), fibroblast growth factor (FGF), and so forth. One of the important characteristics common to each substance is that each such substance is known or believed to enhance cell or tissue growth. Other cell proliferating agents have also been reported such as, for example, those described in EP 0953354, JP 09-227413, JP 53-062815 A2 and EP 0560845 A1. However, there exists a continuing need for additional and/or improved cell proliferating agents and compounds. In particular, there is a continuing need for compounds that promote controlled cell growth without promoting or inducing deleterious or uncontrolled cell growth such as, for example, cancerous growth.

Flavonoids are active molecules having a variety of important functions within the plants they are found. By way of example, certain flavonoids are believed to act as anti-fungal agents, anti-bacterial agents, UV-B protecting agents, and so forth. In addition, certain flavonoids have been found to have various medicinal applications such as, for example anti-virals, anti-inflammatories, antihistamines, anti-cancers and anti-oxidants. These and other uses or functions are described in the following books and articles: B. Bohm, Introduction To Flavonoids Chapters 7-8 (1998); J. Harbourne et al., Advances In Flavonoid Research Since 1992, 55 Phytochemistry 481-500 (2000); A. L. Miller, Antioxidant Flavonoids: Structure, Function and Clinical Usage, Alternate Medicine Review, 1, 103-111 (1996); R. Nijveldt et al., Flavonoids: A Review Of Probable Mechanisms Of Action And Potential Applications, American Journal Of Clinical Nutrition, 74, 418-425 (2001). Previous studies have focused significantly on the inhibitory action of flavonoids against enzyme systems, inflammation and cancer see, for example, J. Harborne, Editor; The Flavonoids: Advances in Research Since 1986, (1994). Moreover, flavones, flavonols, flavanones and isoflavones have been shown to possess anti-proliferative activity for numerous cell lines in vitro. See, S. Kuntz et al., Comparative Analysis Of The Effects Of Flavonoids On Proliferation, Cytotoxicity And Apoptosis In Human Colon Cancer Cell Lines, European Journal Of Nutrition 38, 133-142 (1999). The authors of the aforesaid article demonstrated that this inhibition was not a consequence of cytotoxic effects and concluded that these four classes of flavonoids inhibit in vitro growth by cell cycle arrest.

### SUMMARY OF INVENTION

It has been surprisingly found that flavonoids, when used in the proper dosages and/or amounts, significantly increase cell proliferation and thus can be used to treat wounds as well as other conditions or maladies where increased cell growth would be beneficial. In one aspect, a cell proliferating composition is provided comprising a therapeutically effective amount of a flavonoid selected from the group consisting of flavones, flavonols, flavanones, isoflavanones and isoflavones. In one embodiment, flavonoid is present in the composition in an amount between about 0.00001% and about 5% (by weight). In a specific embodiment, the flavonoid is selected from the group consisting of quercetin, rutin, naringin, hesperidin, silybin, daidzin, genistin and genistein. In a further embodiment, the cell proliferating composition can include one or more pharmaceutical carriers.

In an additional embodiment, a composition for treating wounds is provided comprising (i) an effective amount of a flavonoid selected from the group consisting of flavones, flavonols, flavanones, isoflavanones, and isoflavones; and (ii) a pharmaceutical carrier. In one aspect, the flavonoid is present in an amount between about 0.00001% and about 5% (by weight) of the composition. In a further aspect, the flavonoid is present in an amount sufficient to increase fibroblast cell growth at least 2%. In one embodiment, the pharmaceutical carrier is selected from the group consisting of ointments, creams, gels, foams, sprays, salves, films, and fabrics. In a further embodiment, the composition is a semi-solid material and includes a base selected from the group consisting of hydrocarbon bases, absorption bases, water-removable bases and water-soluble bases. In still a further embodiment, the composition can include one or more active agents selected from the group consisting of emollients, ant-infective agents, preservatives, pH modifiers, mechanical protectants, chemical protectants, adsorbents and humectants. In a further aspect, a method of treating wounds and/or increasing fibroblast cell proliferation is provided comprising the steps of applying to and/or treating tissue containing fibroblast cells with one of the pharmaceutical compositions described herein.

### DESCRIPTION OF THE INVENTION

Reference will now be made in detail to embodiments of the present invention, various specific examples of which will be discussed herein. Each embodiment is provided by way of explanation of the invention, and not meant as a limitation of the invention. For example, features illustrated or described as part of one embodiment may be used with another embodiment to yield still further embodiments. It is intended that the present invention include these and other modifications and variations as come within the spirit of the invention. In addition, throughout this disclosure various theories or mechanisms relating to the present invention are provided. However, the inventor does not wish to be bound by the same and the theories or mechanisms are provided solely to better understand the present invention and are not intended to limit the effective scope of the claims. Further, as used herein, the term “comprising” is inclusive or open-ended and does not exclude additional unrecited elements, compositional components, or method steps. Accordingly, the term “comprising” encompasses the more restrictive terms “consisting essentially of” and “consisting of.”

As indicated above, certain flavonoids have been found to have a cell proliferating effect on mammalian cells and, in particular, upon the proliferation of connective tissue cells such as, for example, fibroblasts. Desirably, the cells and conditions to be treated with the therapeutic compositions and methods of the present invention are those of mammals. Mammals include various classes and families of animals including, but not limited to, primates, bovines, canines, equines, felines, etc. As specific examples, mammals include humans, certain farm animals (e.g. cattle, horses, pigs, etc.), certain lab animals (e.g. mice, rats, rabbits, etc.), many pets and zoo animals (e.g. dogs, cats, monkeys, etc.).

The compositions and methods described herein are believed generally suitable for use in treating conditions where cell proliferation is desirable. By way of non-limiting example, increased proliferation of fibroblasts would be highly beneficial in the treatment of wounds. As a further example, increased proliferation of fibroblast cells would be beneficial in the treatment of the skin by improving the extra-cellular matrix thereby tightening and/or strengthening the skin. More particularly, collagen, the predominant matrix skin protein, is known to impart tensile strength to skin. It has been shown that collagen is significantly reduced with age and UV exposure. The degradation or destruction of the architecture of these proteins decreases the tensile strength of the skin causing wrinkles and laxity. Many studies involving human subjects have shown that collagen type I is decreased with increasing severity of photodamage. See, for example, A. Kligman, Early Destructive Effect Of Sunlight On Human Skin, JAMA, 210, 2377-2380 (1969); R. Lavker, Structural Alterations In Exposed And Unexposed Aged Skin, Journal of Inv. Derm., 73, 59-66 (1979); J. Smith et al., Journal of Investigative Dermatology, 39, 347-350 (1962); and S. Shuster et al., The Influence Of Age And Sex On Skin Thickness, Skin Collagen And Density, British Journal of Dermatology, 93, 639-643 (1975). In addition, some correlation in the histology of wrinkles and reduction in collagen levels in the sun-exposed skin has been reported. See, for example, S. Chen et al., Effects of All-Trans Retinoic Acid on UVB-Irradiated and Non-Irradiated Hairless Mouse Skin, Journal of Investigative Dermatology 98, 248-254 (1992). The restoration of collagen type I in photo-damaged human skin by a topical treatment has also been reported. See, for example, C. Griffiths, et al., Restoration of Collagen Formation in Photodamaged Human Skin by Tretinoin (Retinoic Acid), The New England Journal of Medicine, 329, 530-535 (1993). Thus, it is believed that the cell proliferating compositions and methods of the present invention would also be beneficial to the repair and/or prevention of cutaneous tissue damage associated with exposure to the elements as well as time itself due to the ability to increase the number and/or potency of fibroblasts.

As indicated above, the present invention also provides methods and therapeutically effective compositions for treating wounds. The wounds can be external or internal and as used herein the term “wound” includes tissue that has been incised, lacerated, perforated, abraded, burnt or otherwise degraded. Within the larger class of wounds are acute wounds, chronic wounds, minor cuts and burns. As a particular example, the therapeutic compositions and methods of the present invention can be used to promote the healing of wounds in cutaneous and/or subcutaneous tissues. Epidermal, dermal and underlying subcutaneous tissues suffer from various wounds and healing can be improved in any or all of these tissues utilizing the therapeutic compositions and methods of the present invention. The increased proliferation of fibroblasts, endothelial cells and/or keratinocytes increases the availability of fibronectin and other proteinaceous components which are necessary for the production of collagen, elastin and glycosaminglycans. In addition, it is noted that collagen is a major component of connective tissue matrices, not only in skin, but also in other tissues such as, for example, lungs, bone, synovium, eye, tendons, cartilage and gingiva. In this regard, there is a high correlation between proliferation of fibroblasts and tissue healing. Thus, while the invention is often described with relation to wound healing and/or general strengthening of coetaneous and subcutaneous tissue, comparable beneficial cell proliferation effects would be expected in other cells and tissues and in particular those containing fibroblasts and/or collagen. Therefore, the therapeutic compositions and methods of the present invention are believed useful in treating any traumatized or degraded body tissue in which the increased growth of fibroblasts, epithelial cells or similar cells is beneficial to or otherwise improves healing and/or maintenance of the tissue.

Flavonoids

As indicated above, the present invention relates to the use of therapeutically effective compositions and methods comprising certain flavonoids in order to increase cell proliferation and thereby treat and/or prevent various maladies. Flavonoids are aromatic, heterocyclic compounds commonly found in many higher plants. Flavonoids occur naturally in various parts of plants and, by way of example only, are commonly found in fruits, vegetables, flowers, leaves, bark, roots and so forth. Flavonoids are commonly associated with those compounds within plants that impart color thereto, e.g. yellows, reds, blues, and so forth. However, flavonoids generally refer to the thousands of compounds, whether synthetically manufactured or naturally found within plants, that have a similar skeleton structure. In this regard, many flavonoids (i.e. flavones, flavonols, flavanones, isolfavones, and isoflavanones) comprise a O-heterocyclic ring fused to an aromatic ring with a third ring system attached at either the C-2 or C-3 of the third heterocyclic ring (see Formulas I-IV below). Some examples include genistein, found in soybeans and some other legumes; quercetin, found in apples and onions; PCOs (procyanidolic oligomers, also known as proanthocyanidins), found in abundance in pine bark and grape seed extract, as well as in red wine; citrus flavonoids, including rutin and hesperidin, found in oranges, grapefruits, tangerines and other citrus fruits; and polyphenols, particularly EGCG (epigallocatechin-gallate), found in green tea as well as legumes, grains and nuts. More detail regarding the history, source, uses as well as flavonoid structure can be found in B. Bohm, Introduction To Flavonoids, (1998). The particular flavonoids suitable for use in the present invention include those in the following subclasses: flavones, isoflavones, isoflavanones, flavanones, flavonols and derivatives thereof. Flavonoids are commercially available from various manufacturers including, for example, Indofine Chemical Company, Inc., Sigma Chemical Co., and Aldrich Chemical Co.

Flavones are generally characterized as having a closed three-carbon bridge (i.e. a third ring structure) wherein the second or B-ring is attached at the carbon adjacent the ketone (i.e. the C-2 carbon) and as further having a double bond between the C-2 and C-3 carbons. Flavonols comprise an additional subclass of flavonoids and, while similar to flavones, differ from flavones in that they have a hydroxyl group at the middle carbon of the three-carbon bridge (i.e. the C-3 carbon). By way of non-limiting example, flavones and flavonols believed suitable for use in the present invention include, but are not limited to, the following: Acacetin; Amentoflavone; Sciadopitysin; Apigenin; Aspigetrin; Apiin; Avicularin; Baicalein; Baicalein trimethyl ether; Baicalein-7-O-glucuronide; b-Naphthoflavone; a-Naphthoflavone; 3′-Benzyloxy-5,7-dihydroxy-3,4′-dimethoxyflavone; 3′-Benzyloxy-5,6,7,4′-tetramethoxyflavone; 6-Bromo-4′-chloroflavone; 6-Bromoflavone; 8-Carboxy-3-methylflavone; 4′-Chloro-6,8-dibromoflavone; 4′-Chloroflavone 6-Chloroflavone; 4′-Chloro-6-methylflavone; 6-Chloro-7-methylflavone; Chrysoeriol; Cupressuflavone; Datiscetin; Datiscoside; 6,8-Dibromoflavone; 6,4′-Dichloroflavone; 6,8-Dichloroflavone; 6,4′-Dichloro-7-methylflavone; 3,7-Dihydroxy-3′,4′-dimethoxyflavone; 3,5-Dihydroxyflavone; 3,6-Dihydroxyflavone; 3,7-Dihydroxyflavone; 3,2′-Dihydroxyflavone; 3,3′-Dihydroxyflavone; 5,7-Dihydroxyflavone; 5,2′-Dihydroxyflavone 5,3′-Dihydroxyflavone; 5,4′-Dihydroxyflavone; 6,7-Dihydroxyflavone; 6,2′-Dihydroxyflavone; 6,3′-Dihydroxyflavone; 6,4′-Dihydroxyflavone; 7,8-Dihydroxyflavone; 7,2′-Dihydroxyflavone; 7,3′-Dihydroxyflavone; 7,4′-Dihydroxyflavone; 2′,3′-Dihydroxyflavone; 2′,4′-Dihydroxyflavone; 3′,4′-Dihydroxyflavone; Baicalein-7-methyl ether; Genkwanin; 3′,4′-Dihydroxy-a-naphthoflavone; 3′,4′-Dihydroxy-b-naphthoflavone; Gossypetin; Robinetin trimethyl ether; Eupatorin; 5,7-Dihydroxy-3′,4′,5′-trimethoxyflavone; 3,5-Dimethoxyflavone; 3,6-Dimethoxyflavone; 3,7-Dimethoxyflavone; 3,2′-Dimethoxyflavone; 3,3′-Dimethoxyflavone; 3,4′-Dimethoxyflavone; 5,7-Dimethoxyflavone; 5,2′-Dimethoxyflavone; 5,3′-Dimethoxyflavone; 5,4′-Dimethoxyflavone; 6,7-Dimethoxyflavone; 6,2′-Dimethoxyflavone; 6,3′-Dimethoxyflavone; 6,4′-Dimethoxyflavone; 7,8-Dimethoxyflavone; 7,2′-Dimethoxyflavone; 7,3′-Dimethoxyflavone; 7,4′-Dimethoxyflavone; 2′,3′-Dimethoxyflavone; 2′,4′-Dimethoxyflavone; 3′,4′-Dimethoxyflavone; 3-Hydroxy-3′,4′-dimethoxyflavone; 2′,3′-Dimethoxy-3-hydroxyflavone; 2′,4′-Dimethoxy-3-hydroxyflavone; 3′,4′-Dimethoxy-a-naphthoflavone; 3′,4′-Dimethoxy-b-naphthoflavone; 3,4′-Dimethoxy-5,7,3′-trihydroxyflavone; Diosmetin Diosmin; Eupatorin-5-methyl ether; Fisetin; Flavone; Fortunellin; Galangin; Gardenin; Geraldol; Gossypetin; Gossypin; 5,6,7,3′,4′,5′-Hexamethoxyflavone; Hinokiflavone; Homoorientin; Scutellarein; Eucalyptin; 3-Hydroxy-6,4′-dimethoxyflavone; 3-Hydroxy-7,4′-dimethoxyflavone; 3-Hydroxy-2′,4′-dimethoxy-6-methylflavone; 3-Hydroxyflavone (Flavonol); Primuletin; 6-Hydroxyflavone; 7-Hydroxyflavone; 2′-Hydroxyflavone; 3′-Hydroxyflavone; 4′-Hydroxyflavone; 6-Hydroxyflavone-b-D-glucoside; 7-Hydroxyflavone-b-D-glucoside; 3-Hydroxy-5-methoxyflavone; 3-Hydroxy-6-methoxyflavone; 3-Hydroxy-7-methoxyflavone; 3-Hydroxy-2′-methoxyflavone; 3-Hydroxy-3′-methoxyflavone; 3-Hydroxy-4′-methoxyflavone; Tectochrysin; 5-Hydroxy-2′-methoxyflavone; 5-Hydroxy-3′-methoxyflavone; 5-Hydroxy-4′-methoxyflavone; 6-Hydroxy-7-methoxyflavone; 6-Hydroxy-2′-methoxyflavone; 6-Hydroxy-3′-methoxyflavone; 6-Hydroxy-4′-methoxyflavone; 7-Hydroxy-2′-methoxyflavone; 7-Hydroxy-3′-methoxyflavone; Pratol; 8-Hydroxy-7-methoxyflavone; 4′-Hydroxy-5-methoxyflavone; 4′-Hydroxy-6-methoxyflavone; 4′-Hydroxy-7-methoxyflavone; 4′-Hydroxy-3′-methoxyflavone; 3-Hydroxy-4′-methoxy-6-methylflavone; 3-Hydryoxy-6-methylflavone; 7-Hydroxy -3-methylflavone; 7-Hydroxy-5-methylflavone; 2′-Hydroxy-a-naphthoflavone; 2′-Hydroxy-b-naphthoflavone; 4′-Hydroxy-a-naphthoflavone; 4′-Hydroxy-b-naphthoflavone; Quercetin tetramethyl ether; 3′-Hydroxy-5,6,7,4′-tetramethoxyflavone; 3-Hydroxy-3′,4′,5′-trimethoxyflavone; 3-Hydroxy-6,2′,3′-trimethoxyflavone 3-Hydroxy-6,2′,4′-trimethoxyflavone; 3-Hydroxy-6,3′,4′-trimethoxyflavone; 3-Hydroxy-7,2′,3′-trimethoxyflavone; 3-Hydroxy-7,2′,4′-trimethoxyflavone; Hyperin; Isoquercitrin; Isorhamnetin; Isorhamnetin-3-glucoside; Narcisin; Isorhoifolin; Isovitexin; Kaempferide; Kaempferol; Astragalin; Kaempferol-7-neohesperidoside; Kaempferol-3-rutinoside; Kaempferol-3,7,4′-trimethylether; Karanjin; Linarin; Liquiritigenin; Luteolin; Luteolin; Luteolin-7,3′-diglucoside; Luteolin-4′-glucoside; Luteolin-7-glucoside; Maritimein; 3-Methoxyflavone; 5-Methoxyflavone; 6-Methoxyflavone; 7-Methoxyflavone; 2′-Methoxyflavone; 3′-Methoxyflavone; 4′-Methoxyflavone; 4′-Methoxyflavonol; 6-Methoxyluteolin; 2′-Methoxy-a-naphthoflavone; 2′-Methoxy-b-naphthoflavone; 4′-Methoxy-a-naphthoflavone; 6-Methylflavone; 8-Methylflavone; 6-Methyl-4′-methoxyflavone; 8-Methyl-4′-methoxyflavone; Morin; Myricetin; Myricitrin; Neodiosmin; Orientin; Peltatoside; Robinetin; Sinensetin; 5,7,3′,4′,5′-Pentamethoxyflavone; Quercetagetin; Quercetin; Quercetin-3-O-b-D-glucopyranosyl-6″-acetate; Quercetin-3,5,7,3′,4′-pentamethyl ether; 3,5,7,3′,4′-Pentamethoxyflavone; Quercetin-3-O-sulfate potassium salt; Retusin; Quercitrin; Rhoifolin; Robinin; Rutin; Saponarin; Scutellarein tetramethyl ether; Spiraeoside; Sulfuretin; Syringetin-3-galactoside; Syringetin-3-glucoside; Tamarixetin; Syringetin; 3,6,2′,4′-Tetrahydroxyflavone; 7,8,3′,4′-Tetrahydroxyflavone; Rhamnetin; 3,6,3′,4′-Tetramethoxyflavone; 5,7,3′,4′-Tetramethoxyflavone; 7,8,3′,4′-Tetramethoxyflavone; Tiliroside; 6,8,4′-Trichloroflavone; 3,6,4′-Trihydroxyflavone; 3,7,4′-Trihydroxyflavone; 3,3′,4′-Trihydroxyflavone; 5,7,8-Trihydroxyflavone; 5,7,2′-Trihydroxyflavone; 5,3′,4′-Trihydroxyflavone; 6,3′,4′-Trihydroxyflavone; 7,8,2′-Trihydroxyflavone; 7,8,3′-Trihydroxyflavone; 7,8,4′-Trihydroxyflavone; 7,3′,4′-Trihydroxyflavone; 3,5,7-Trihydroxy-3′,4′,5′-trimethoxyflavone; 5,7,4′-Trimethoxyflavone; 7,3′,4′-Trimethoxyflavone; Vitexin; Vitexin-2″-O-rhamnoside and so forth.

Isoflavones comprise another class of flavonoids and they are generally characterized by having a closed three-carbon bridge (i.e. a third ring structure) with the second or B-ring attached at the middle carbon of the three-carbon bridge (i.e. the C-3 position) and as further having a double bond between the C-2 and C-3 carbons. By way of non-limiting examples, isoflavones believed suitable for use with the present invention include, but are not limited to, the following: Daidzin; Daidzein; Biochanin A; Prunetin; 7,4′-Dimethoxyisoflavone; Equol; Genistin; Glycitein; Glycitin; 5-Hydroxy-7,4′-dimethoxyisoflavone; Formononetin; Formonetin; Ononin; Osajin; Pomiferin; Puerarin; Sissotrin; Genistein; 6,7,4′-Trihydroxyisoflavone; 7,3′,4′-Trihydroxyisoflavone; 6,7,4′-Trimethoxyisoflavone and so forth.

Flavanones comprise a still another class of flavonoids and they are generally characterized as having a closed, saturated three-carbon bridge with the second or B-ring attached at the carbon adjacent the ketone (i.e. the C-2 position). By way of non-limiting examples, flavanones believed suitable for use with the present invention include, but are not limited to, the following: Bavachinin A; Didymin; Dihydrorobinetin; Pinocembrin; Sakuranetin; 5,7-Dihydroxy-3′,4′,5′-trimethoxyflavanone; 2′,3′-Dimethoxyflavanone; 5,7-Dimethoxyflavanone Eriocitrin; Eriodictyol; Eriodictyol; Eriodictyol-7-glucoside; Neoeriocitrin; Flavanomarein; Flavanone (2,3-Dihydroflavone); Flavanone azine; Flavanone diacetylhydrazone; Flavanone hydrazone; Hesperetin; Neohesperidin; Hesperidin; Homoeriodictyol; 4′-Hydroxy-5,7-dimethoxyflavanone; 6-Hydroxyflavanone; 7-Hydroxyflavanone; 2′-Hydroxyflavanone; 3′-Hydroxyflavanone; Pinostrobin; Isosakuranetin; 5-Methoxyflavanone; 6-Methoxyflavanone; 7-Methoxyflavanone; 4′-Methoxyflavanone; Naringenin; Naringenin-7-glucoside; Naringin; Narirutin; Taxifolin; Pinostrobin; Poncirin; Silybin; Fustin; Farrerol; 6,2′,3′-Trimethoxyflavanone; 6,3′,4′-Trimethoxyflavanone and so forth.

In a particular embodiment, flavones and flavonols, and including glycosides and other derivatives thereof, are believed suitable for use in the present invention. More particularly, flavones and flavonols having the following formula are believed suitable for use in the present invention:

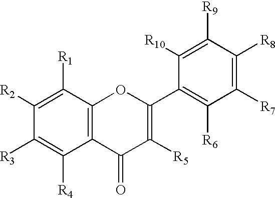

Wherein:

at least one of R₁-R₄ is OH, sugar, or OR (where R is an alkyl or aryl);

at least one of R₆-R is OH sugar, or OR (where R is an alkyl or aryl);

R₁-R₁₀ can be selected from H; OH; halide; CHO; OR (where R is an alkyl or aryl); alkyl with C1-C20; COOR (where R is an alkyl or aryl); COR (where R is an alkyl or aryl); amine; amide, RCONH2 (where R is an alkyl or aryl); RCONRₐR_{b} (where Rₐ is H or alkyl C₁-C₂₀, and R_{b}=H or alkyl C₁-C₂₀); aryl (substituted or unsubstituted).

With regard to the foregoing, in one embodiment the alkyl groups extending from one of the three rings forming the flavone or from a functional group thereon, have less than 20 carbons. In a further embodiment, the sugar can include mono, di and trisacharides selected from hexose and pentose. Still further, the sugar moiety can be directly attached to one of the three main rings or via a C or O, e.g. C-glycosides or O-glycosides.

In a further embodiment, flavanones, including glycosides and other derivatives thereof, are believed suitable for use in the present invention. More particularly, flavanones having the following formula are believed suitable for use in the present invention:

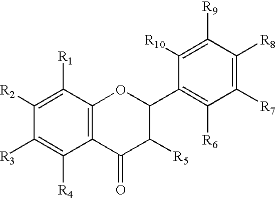

Wherein:

at least one of R₁-R₄ is OH, sugar, or OR (where R is an alkyl or aryl);

at least one of R₆-R₁₀ is OH sugar, or OR (where R is an alkyl or aryl);

R₁-R₁₀ can be selected from H; OH; halide; CHO; OR (where R is an alkyl or aryl); alkyl with C1-C20; COOR (where R is an alkyl or aryl); COR (where R is an alkyl or aryl); amine; amide, RCONH2 (where R is an alkyl or aryl); RCONRₐR_{b} (where Rₐ is H or alkyl C₁-C₂₀, and R_{b}=H or alkyl C₁-C₂₀); aryl (substituted or unsubstituted).

With regard to the foregoing, in one embodiment the alkyl groups extending from one of the three rings forming the flavone or from a functional group thereon, have less than 20 carbons. In a further embodiment, the sugar can include mono, di and trisacharides selected from hexose and pentose. Still further, the sugar moiety can be directly attached to one of the three main rings or via a C or O, e.g. C-glycosides or O-glycosides.

In a further embodiment, isoflavones, including glycosides and other derivatives thereof, are believed suitable for use in the present invention. More particularly, isoflavones having the following formula are believed suitable for use in the present invention

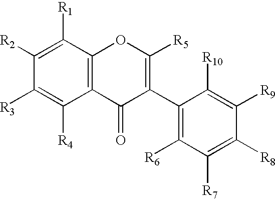

Wherein:

at least one of R₁-R₄ is OH, sugar, or OR (where R is an alkyl or aryl);

at least one of R₆-R₁₀ is OH sugar, or OR (where R is an alkyl or aryl);

R₁-R₁₀ can be selected from H; OH; halide; CHO; OR (where R is an alkyl or aryl); alkyl with C1-C20; COOR (where R is an alkyl or aryl); COR (where R is an alkyl or aryl); amine; amide, RCONH2 (where R is an alkyl or aryl); RCONRₐR_{b} (where Rₐ is H or alkyl C₁-C₂₀, and R_{b}=H or alkyl C₁-C₂₀); aryl (substituted or unsubstituted).

With regard to the foregoing, in one embodiment the alkyl groups extending from one of the three rings forming the flavone or from a functional group thereon, have less than 20 carbons. In a further embodiment, the sugar can include mono, di and trisacharides selected from hexose and pentose. Still further, the sugar moiety can be directly attached to one of the three main rings or via a C or O, e.g. C-glycosides or O-glycosides.

In a further embodiment, isoflavanones, including glycosides and other derivatives thereof, are believed suitable for use in the present invention. More particularly, flavanones having the following formula are believed suitable for use in the present invention:

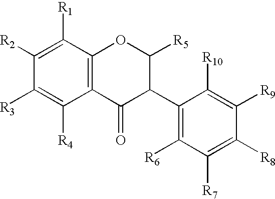

Wherein:

at least one of R₁-R₄ is OH, sugar, or OR (where R is an alkyl or aryl);

at least one of R₆-R₁₀ is OH sugar, or OR (where R is an alkyl or aryl);

R₁-R₁₀ ran be selected from H; OH; halide; CHO; OR (where R is an alkyl or aryl); alkyl with C1-C20; COOR (where R is an alkyl or aryl); COR (where R is an alkyl or aryl); amine ; amide, RCONH2 (where R is an alkyl or aryl); RCONRₐR_{b} (where Rₐ is H or alkyl C₁-C₂₀, and R_{b}=H or alkyl C₁-C₂₀); aryl (substituted or unsubstituted).

With regard to the foregoing, in one embodiment the alkyl groups extending from one of the three rings forming the flavone or from a functional group thereon, have less than 20 carbons. In a further embodiment, the sugar can include mono, di and trisacharides selected from hexose and pentose. Still further, the sugar moiety can be directly attached to one of the three main rings or via a C or O, e.g. C-glycosides or O-glycosides.

While it has been found that flavonoids can be used to increase mammalian cell proliferation and, as a specific example increase fibroblast proliferation, it is important to note the well documented and known inhibitory effects of flavonoids. In this regard it has been found that the cell proliferation effects of flavonoids are dose dependent. When flavonoids are utilized to treat tissues and cells in certain ranges an inhibitory effect, i.e. a decrease in cell growth or proliferation, can be seen. Thus, the compositions and methods of the present invention utilize flavonoids in a therapeutically effective amount to increase cell proliferation. In a particular embodiment, the flavonoids are utilized in a therapeutically effective amount to improve healing in tissues such as cutaneous tissue and other tissue. As used herein an “effective amount” or a “therapeutically effective amount” refers to an amount that is sufficient to increase cell proliferation. In this regard, increased cell proliferation is relative to normal cell growth rates for like tissue, i.e. similar in age, nature or degree of damage, etc. In a particular embodiment, the desired tissues and/or cells are treated with one or more of the aforesaid flavonoid compounds in an amount sufficient to increase cell growth rate by more than 2%. Desirably, the tissues and/or cells are treated with one or more of the aforesaid flavonoid compounds in an amount sufficient to increase cell growth rate at least about 5% and still more desirably at least about 10% or more. In a particular embodiment, cutaneous, subcutaneous and/or other tissues are treated with one or more of the aforesaid flavonoid compounds in an amount sufficient to increase fibroblast growth rates at least 2% and, still more desirably, in an amount sufficient to increase fibroblast growth rate at least about 5% and, even still more desirably, in an amount sufficient to increase fibroblast growth rate at least about 10%.

Pharmaceutical Preparations and Compositions

The therapeutically effective compositions of the present invention can be applied systemically, topically, intramuscularly, and/or locally. The therapeutically effective compositions may be stored for future use or may be formulated in effective amounts within pharmaceutically acceptable carriers to prepare a wide variety of pharmaceutical compositions. Examples of pharmaceutically acceptable carriers are pharmaceutical appliances, topical vehicles (non-oral and oral), ingestible vehicles and so forth. In addition, the pharmaceutical compositions of the present invention can be made using manufacturing techniques and processes readily known to those skilled in the art.

Examples of pharmaceutical appliances are sutures, staples, gauze, bandages, burn dressings, artificial skins, liposome or micell formulations, microcapsules, aqueous articles for soaking gauze dressings, and so forth. As specific examples thereof, various pharmaceutical appliances suitable for use with the compositions of the present invention are described in Remington: The Science and Practice of Pharmacy, 20th Edition, Editors, A. R. Gennaro et al., Lippincott Williams & Wilkins, PA, USA (2000).

In addition, ingestible compositions desirably can employ ingestible or partly ingestible vehicles such as confectionery bulking agents which include hard and soft vehicles such as, for example, tablets, suspensions, chewable candies or gums, lozenges and so forth. Exemplary ingestable vehicles and compositions for use therein are described in Remington: The Science and Practice of Pharmacy, 20^{th} Edition (Lippincott Williams & Wilkins 2001); Goodman & Gilman's The Pharmaceutical Basis of Therapeutics, 10^{th} Edition (McGraw-Hill Professional, 2001) and The United States Pharmacopoeia: The National Formulary (US Pharmacopoeia Convention, Inc.).

Topical compositions may employ one or more carriers or vehicles such as, for example, creams, gels, foams, ointments, sprays, salves, bio-adhesives, films, fabrics and so forth, which are intended to be applied to the skin or a body cavity. Topical compositions may also be adapted for use as an oral vehicle such as, for example, mouthwashes, rinses, oral sprays, suspensions, and dental gels, which are intended to be taken by mouth but are not intended to be ingested. Exemplary topical compositions and components thereof are described in Remington: The Science and Practice of Pharmacy, 20th Edition, Editors, A. R. Gennaro et al., Lippincott Williams & Wilkins, PA, USA (2000); Goodman & Gilman's The Pharmacological Basis of Therapeutics, 10th Edition, Editors, J. G. Hardman and L. E. Limbird., McGraw-Hill Professional, (2001). The United States Pharmacopoeia: The National Formulary, United States Pharmacopoeial Convention, Inc., Rockville, Md. Topical ointments and other semi-solid compositions commonly employ one or more bases as a vehicle for drug delivery. Exemplary bases include, but are not limited to, hydrocarbon bases (e.g. white petrolatum, white ointment, vegetable oils, animals fats, etc.), absorption bases (e.g. hydrophilic petrolatum, anhydrous lanolin, lanolin, cold cream, etc.), water-removable bases (e.g. hydrophilic ointment USP, ethoxylated fatty alcohol ethers, ethoxylated lanolin derivatives, sorbitan fatty acid esters, etc.), and water-soluble bases (e.g. polyethylene glycol ointment, etc.). As further specific examples thereof, topical compositions believed suitable for use with the present invention are described in U.S. Pat. No. 6,046,160, the entire contents of which are incorporated herein by reference

A variety of traditional ingredients may optionally be included in the pharmaceutical compositions in effective amounts. By way of non-limiting example, the pharmaceutical compositions can contain one or more of the following materials: fillers, diluents, cleaning agents, buffers, preservatives, pH and toxicity modifiers, mechanical protectants, chemical protectants, adsorbents, antioxidants, viscosity modifiers, extenders, excipients, astringents, emollients, demulcents, humectants, emulsifiers, transdermal delivery enhancing agents, controlled-release agents, dyes or colorants, stabilizers, lubricants and so forth. These and other conventional pharmaceutical additives known to those having ordinary skill in the pharmaceutical arts can be used in the pharmaceutical composition as dictated by the nature of the delivery vehicle.

The amounts of additional components within the compositions are readily determined by those skilled in the art without the need for undue experimentation and will vary with the nature of the vehicle (e.g. a gel versus a spray), the wound to be treated, frequency of treatment and so forth. Thus, the amount of therapeutic wound healing composition may be varied in order to obtain the result desired in the final product and such variations are within the capabilities of those skilled in the art without the need for undue experimentation. In a particular embodiment, the pharmaceutical composition can comprise a pharmaceutical composition having one or more flavonoids present in an amount less than 50% and in a further embodiment in an amount less than about 20% by weight of the pharmaceutical composition. In a further embodiment, the pharmaceutical compositions can contain one or more of the aforesaid flavonoids in an amount between about 0.00001% to about 5%, by weight of the pharmaceutical composition. In an alternate embodiment, the pharmaceutical composition comprises one or more of the aforesaid flavonoids in an amount between about 0.001% to about 1%, by weight of the pharmaceutical composition.

Test Methods

The proliferative response of flavonoids to the human skin fibroblast cell line (ATCC, Manassas, Va., cell line CRL-2522 human foreskin fibroblast) was determined in a 96-well assay system using serum-free medium as a control. Stock solutions of flavonoids were prepared as 1.0 mole/L solutions in Dimethyl sulfoxide (DMSO, Sigma Chemical Company, St. Louis, Mo.) then diluted with Dulbecco's Modified Eagle's Medium (DMEM, Sigma Chemical Co., St. Louis, Mo.) to 10⁻⁴, 10⁻⁵, and 10⁻⁶ M solutions with a final DMSO concentration of 0.1% (v:v). Cells were seeded into 96 well plates at a concentration of 2×10³ cells in 100 μl of DMEM containing 10% fetal bovine serum (FBS, Sigma Chemical Co., St. Louis, Mo.). Plates were incubated for 24 hours at 37° C. in a humidified, 5% CO₂ atmosphere. After incubation, the medium was aspirated and the wells were rinsed twice with 100 μl of serum-free DMEM. The final rinse was aspirated and 100 μl of the 10⁻⁴-10⁻⁶ M of each solution was added to 6 wells. In addition, 100 μl of vehicle (serum free DMEM with DMSO at 0.1% v:v) was added to 6 wells as control. In addition, duplicate wells of each test and control solution were placed in wells without the human fibroblast cells to serve as blanks for each treatment. All wells were incubated for 28 hours at 37° C. in a humidified, 5% CO₂ atmosphere. After incubation, 20 μl of Cell Titer 96 Aqueous One Solution Reagent (Promega, Corp., Madison, Wis.) was added to all wells. The plates were swirled gently and place back in the incubator for 45 minutes and spectrophotometric absorbance was read at 490 nm. Absorbance of the wells without cells was subtracted from the absorbance of the wells with cells to give the true absorbance.

Cell growth rates for other cell lines may be determined in a similar manner. However, one skilled in the art will appreciate that various aspects of the test will change in accord with the particular cell line being evaluated.

### EXAMPLES

Various compounds of present invention were tested for their cell proliferating effect on human skin fibroblasts. The effect of flavonoids naringin and quercetin on cell proliferation in human foreskin fibroblasts (ATCC cell line CRL-2522) was measured and data is presented in FIG. 1. The control and all test materials contained a final concentration of 0.1% DMSO in DMEM with n=6. Sample concentrations were 10⁻⁴, 10⁻⁵ and 10⁻⁶ M.

Table 1

[image]

Statistical analysis was done by using one-way ANOVA. Statistically significant difference (P<0.01 and 0.05) was observed between controls and naringin and controls and quercetin. Based on this significant statistical difference, quercetin and naringin are good cell proliferating agents at lower concentrations. However, at higher concentrations, quercetin appears to show cell inhibition as evidenced by the decrease in cell growth relative to the control

What is claimed is:

1. A cell proliferating composition comprising a therapeutically effective amount of flavonoid selected from the group consisting of flavones, flavonols, flavanones, isoflavanones, isoflavones and derivatives thereof.

2. The cell proliferating composition of claim 1 comprising wherein said flavonoid is present in an amount between about 0.0001% and about 5%.

3. The cell proliferating composition of claim 1 wherein said flavonoid comprises a flavonol.

4. The cell proliferating composition of claim 1 wherein said flavonoid comprises a flavanone.

5. The cell proliferating composition of claim 1 wherein said flavonoid comprises a flavone.

6. The cell proliferating composition of claim 1 wherein said flavonoid comprises an isoflavanone.

7. The cell proliferating composition of claim 1 wherein said flavonoid comprises an isoflavone.

8. The cell proliferating composition of claim 1 wherein said flavonoid is selected from the group consisting of quercetin and rutin.

9. The cell proliferating composition of claim 1 wherein said flavonoid is selected from the group consisting of naringin, hesperidin and silybin.

10. The cell proliferating composition of claim 1 wherein said flavonoid is selected from the group consisting of daidzin, genistin and genistein.

11. The cell proliferating composition of claim 1 further including a pharmaceutical carrier.

12. A composition for treating wounds comprising:
(i) an effective amount of flavonoid selected from the group consisting of flavones, flavonols, flavanones, isoflavanones, isoflavones and derivatives thereof; and
(ii) a pharmaceutical carrier.

13. The composition of claim 12 comprising wherein said flavonoid is present in an amount between about 0.00001% and about 10%.

14. The composition of claim 12 wherein said flavonoid comprises a flavonol

15. The composition of claim 12 wherein said flavonoid comprises a flavanone.

16. The composition of claim 12 wherein said flavonoid comprises a flavone.

17. The composition of claim 12 wherein said flavonoid comprises an isoflavanone.

18. The composition of claim 12 wherein said flavonoid comprises an isoflavone.

19. The composition of claim 13 wherein said carrier is selected from the group consisting of ointments, creams, gels, foams, sprays, salves, films, and fabrics.

20. The composition of claim 12 wherein said composition is a semi-solid material and includes a base selected from the group consisting of hydrocarbon bases, absorption bases, water-removable bases and water-soluble bases.

21. The composition of claim 20 further comprising a at least one active agent selected from the group consisting of emollients, anti-infective agents, preservatives, pH modifiers, mechanical protectants, chemical protectants, adsorbents and humectants.

22. A method of increasing fibroblast cell proliferation comprising:
treating tissue containing fibroblast cells with a therapeutically effective amount of flavonoid selected from the group consisting of flavones, flavonols, flavanones, isoflavanones, isoflavones and derivatives thereof.

23. The method of claim 22 wherein said tissue is treated with a pharmaceutical composition containing said flavonoid and wherein said pharmaceutical composition includes less than about 20%, by weight, of said flavonoid.

24. The method of claim 22 wherein said flavonoid comprises a flavonol.

25. The method of claim 22 wherein said flavonoid comprises a flavanone.

26. The method of claim 22 wherein said flavonoid comprises a flavone.

27. The method of claim 22 wherein said flavonoid comprises an isoflavone.

28. The method of claim 22 wherein said flavonoid comprises an isoflavanone.

29. The method of claim 22 wherein said flavonoid comprises one or more flavonoids selected from the group consisting of naringin, hesperidin, silybin, daidzin, genistin, genistein, quercetin and rutin.

30. The method of claim 22 wherein said flavonoid is administered orally, topically, intravenously, intramuscularly, transdermally, transnasally, transmucosally or rectally.

31. The method of claim 22 wherein said tissue comprises cutaneous tissue.

32. The method of claim 31 wherein said flavonoid is treated by topically applying said therapeutically effective amount of flavonoid.

33. A method of treating a wound comprising:
(i) providing a pharmaceutical composition containing a therapeutically effective amount of flavonoid selected from the group consisting of flavones, flavonols, flavanones, isoflavanones, isoflavones and derivatives thereof;
(ii) treating the wound with said pharmaceutically composition.

34. The method of claim 33 wherein said wound comprises an acute wound.

35. The method of claim 33 wherein said wound comprises a chronic wound.

36. The method of claim 33 wherein said wound comprises a burn.

37. The method of claim 33 wherein said flavonoid comprises between about 0.00001% and about 10%, by weight, of said pharmaceutical composition.

38. The method of claim 37 wherein said flavonoid comprises one or more flavonoids selected from the group consisting of naringin, hesperidin, silybin, daidzin, genistin, genistein, quercetin and rutin.
